(19) United States
(12) Patent Application Publication
(10) Pub. No.: US 2009/0047242 A1
(43) Pub. Date: Feb. 19, 2009

(54) CONDITIONED BLOOD COMPOSITION AND METHOD FOR ITS PRODUCTION

(76) Inventors: Julio Reinecke, Koln (DE); Peter Wehling, Dusseldorf (DE)

Correspondence Address: OSTROLENK FABER GERB & SOFFEN, 1180 AVENUE OF THE AMERICAS, NEW YORK, NY, 100368403, US

(21) Appl. No.: 12/162,022

(22) PCT Filed: Feb. 2, 2007
(86) PCT No.: PCT/EP07/00903

§ 371 Date: Jul. 24, 2008

(30) Foreign Application Priority Data
Feb. 3, 2006 (DE) 10 2006 005 016.9

(51) Int. Cl. A61K 35/14; A61K 38/20; A61P 21/00; A61P 37/00; A61P 17/06

(52) U.S. Cl. 424/85.2; 424/529

(57) ABSTRACT

The present invention relates to methods for the production of conditioned blood compositions which comprise induced factors, and to conditioned blood compositions preparable by the method and to the use thereof for the treatment or prevention of a disorder of the human or animal body.

The present invention relates to methods for the production of conditioned blood compositions which comprise induced factors or cytokines, and to conditioned blood compositions and to the use thereof for the treatment or prevention of a disorder of the human or animal body.

### PRIOR ART

It is known that blood components, in particular proteins, factors or cytokines such as erythropoietin, insulin or interferon which are present in the blood or blood serum, have no therapeutic or prophylactic activity. Known factors such as the interleukin-1 receptor antagonist (IL-1Ra) inhibit the effect of inflammation-inducing processes. It is further known that such blood components are produced in part by blood tissue itself or are secreted from the blood cells into the plasma phase of the blood.

The production or release of particular blood components such as factors or cytokines can be increased for example by incubation of whole blood taken from an animal or human body. The concentration of certain factors in the incubated blood after incubation is then often higher. The blood components can then be isolated where appropriate. The blood containing the induced factors can also be freed of cellular constituents and be (re)administered as so-called induced blood serum to the human or animal body.

The process of increasing the production or release of blood components such as factors or cytokines is referred to as “induction”. A known method for the induction of blood components in whole blood consists essentially of whole blood being taken from a human or animal body and then incubated in a modified disposable syringe in which special glass beads treated with chromic acid are present, for a particular time under cultivation conditions (Meijer et al. Inflamm. res. 52 (2003): 1-4). The cellular constituents are then removed to result in a conditioned blood serum composition in which some factors or cytokines are induced. In this way, a serum in which the proportion of the antiinflammatory cytokines interleukin-1 receptor antagonist (IL-1Ra), interleukin-4 (IL-4) and interleukin-10 (IL-10) is increased by comparison with freshly removed whole blood is obtained from human venous whole blood. The duration of incubation of the whole blood in this case is 24 hours, and the incubation temperature is about 37° C.

Blood serum compositions produced in this way are employed for the treatment of various inflammatory disorders and autoimmune diseases, for example rheumatoid arthritis. It emerges that, for example, rheumatoid arthritis can be alleviated or cured by a local and/or systemic administration of such conditioned blood serum compositions. The efficacy of the therapy is, however, in need of improvement.

There are in addition further disorders, for example muscle injuries, which can be treated at least in an animal model by local or systemic administration of recombinant cytokines such as IL-1Ra and the like. The blood serum compositions which can be produced in a known manner show only inadequate or no effects in this case. Moreover, muscle injuries are common precisely in the area of sports medicine; they account for up to 30% of the diseases or injuries acquired through sport. More than 90% of these muscle injuries are caused either by contusion or by extreme strain of the muscle. These injuries regularly lead to severe pain and as a result to an inability to continue training or to continue to engage in the sport in the short term or permanently. The state of the art is therefore in need of improvement.

An equine disorder which is to be taken seriously is chronic or periodic eye inflammation (equine recurrent uveitis, ERU). The assumption concerning this chronic inflammation in the current state of the art is that so-called leptospiral allergy, and an acute or chronic leptospiral infection is important for the development of the chronic eye inflammation. The level of infection with these parasitic organisms prevailing in Germany is up to 80%. Various conditions are manifested in some infected animals, including chronic eye inflammation. It moreover appears to be decisive for the development of the disease whether the immune system of the animal tolerates leptospira as parasitic organisms or not.

Horses may develop lameness originating from an extensive inflammation or irritation of the tendon sheath. A further cause of lameness may also be degenerative changes within the tendon tissue, called core lesions. These are likewise followed by extensive inflammatory reactions. The symptoms of inflammations and lameness are ordinarily treated with glucocorticoids (cortisone), cell macerates (ACell®), platelet concentrates (Osteokin®, Magellan® etc.), or else cell preparations from bone marrow or adipose tissue (“stem cells”).

The condition of neurodermatitis is caused by an overreaction of the immune system. However, a therapy with cortisone-containing ointments which is frequently applied at present is associated with some side effects.

In addition, inflammations, or irritations irritations of the nervous system of mostly unknown origin occur frequently in the population. Symptoms frequently occurring in this connection are backaches. Through pain as the cause of inflammation can in this connection frequently be treated only symptomatically by administering analgesics, or by glucocorticoids (such as triamcinolone).

Endometriosis is a disorder in which cells or tissue from the uterine mucosa invade the abdominal cavity and there lead to mostly benign neoplasms. This neoplasm is usually hormone-sensitive and generates severe pain, depending on the hormone status. Surgical resection or hormone treatment are able to provide a remedy for the symptoms associated with these diseases. However, relapses and recurrences are common. Neoplasms may become chronic to such an extent that there is adhesion of further organs and severe chronic pain develops. The symptoms can often be made bearable only with strong analgesics. About 10% of all women develop endometriosis between puberty and menopause and experience symptoms which are more or less severe. An extreme form of this disorder may lead to infertility.

There is thus a need for improved, alternative active substance compositions which can be produced simply, and methods for their production, for effective treatment of the disorders defined above, and further disorders which can be treated by factors or cytokines occurring in the blood. The technical problem underlying the present invention consists in particular of providing an improved method for producing a conditioned blood composition which comprises certain induced factors or cytokines and can be employed effectively for treatment and prevention.

The underlying technical problem is essentially solved by the provision of a method for producing a conditioned blood condition from blood, where the method includes the following steps at least:

In step (a), blood, preferably venous whole blood, is taken from a human or animal body in a manner known per se, preferably freshly by means of venepuncture. In step (b), which preferably follows directly, the removed blood is incubated in at least one modified vessel in order to induce factors or cytokines in the blood composition, that is to say to stimulate the production and release of such factors or cytokines in the blood tissue. The temperature during the incubation of the blood in the modified vessel according to the invention is from 10 to 40° C., preferably from 25 to 40° C., more preferably about 37° C. In step (c), a conditioned blood composition which is rich in certain induced factors or cytokines is obtained in the modified vessel.

For the incubation of the blood there is used according to the invention at least one modified vessel which is characterized in that it has an internal surface area per 1 ml of incubated blood of at least about 100 mm²/ml or more, in particular 104 mm²/ml or more, 123 mm²/ml or more, 131 mm²/ml or more, 224 mm²/ml or more or 283 mm²/ml or more. In a preferred variant, the vessel has an internal surface area of about 200 to about 750 mm²/ml, particularly preferably about 250 to about 650 mm²/ml.

The vessel preferably has a capacity of 5 ml or more, 10 ml or more, 50 ml or more, 60 ml or more, 100 ml or more. If it is intended for example to remove and to incubate an amount of about 50 ml of blood, the internal surface area of the modified vessel should have according to the invention at least about 6600 mm² (66 cm²), preferably about 10 000 mm² to about 37 500 mm² (100 to 375 cm²). If it is intended for example to remove and to incubate an amount of about 10 ml of blood, the internal surface area of the modified vessel should have according to the invention at least about 2300 cm² (23 cm²), preferably about 2500 mm² to about 7500 mm² (25 to 75 cm²).

The “internal surface area” of the vessel means the surface area in the interior of the vessel which is in contact during the incubation with the blood composition to be conditioned, that is to say is essentially wetted thereby.

The invention thus provides for blood which has been taken from a human or animal body to be incubated in a specific modified vessel with a particular surface index of the internal surface area of 200 mm²/ml or more. The inventors have surprisingly found that it is possible by the procedure of the invention to obtain in the modified vessel after a comparatively short time a conditioned blood composition which comprises a high proportion of certain induced factors and in which for example the factor IL-6 is present in high concentration. Moreover, the procedure of the invention surprisingly leads to a blood composition which has high activity prophylactically and therapeutically. Thus, for example, inflammatory joint disorders, eye inflammation in horses, tendon injuries, nerve injuries, endometriosis, neurodermatitis and muscle injuries can be effectively treated by administering the conditioned blood composition obtained according to the invention as blood serum composition into the organism with the disorder or into or onto the organ with the disorder.

It is possible by the procedure of the invention to obtain for example a conditioned blood composition from freshly removed whole blood from human donors in which IL-6 is present in freshly removed a proportion of more than 2000 pg/ml. By comparison therewith, the proportion of IL-6 in unconditioned whole blood is from about 0.5 to about 15 pg/ml. Thus, ordinarily, an approximately 200-fold to approximately 4000-fold increase in the content of IL-6 is achieved according to the invention.

Besides the particularly noteworthy factor IL-6, further therapeutically and prophylactically effective components such as factors or cytokines are obtained in high proportion in the conditioned blood composition. These include besides the known factors IL-4, IL-10 and IL-1Ra surprisingly also factors such as interleukin-13 (IL-13), interleukin-1 (IL-1), especially IL-1β, tumor necrosis factor (TNF), insulin-like growth factor (IGF), transforming growth factor (TGF), platelet-derived growth factor (PDGF), fibroblast growth factor (FGF) and hepatocyte growth factor (HGF). There is thus advantageously a cocktail of different efficiently induced factors or cytokines present in the conditioned blood composition which can be produced according to the invention. Without being bound to the theory, the cocktail of factors and cytokines obtainable according to the invention itself represents the therapeutically and prophylactically highly effective active substance composition.

It is possible in this connection for the abovementioned active substances to be present within the blood composition also in the form of vesicles, microvesicles or exosomes. Vesicles and microvesicles mean subcellular constituents which can inter alia be snared by the membrane surface of immune cells. Exosomes mean subcellular constituents which represent vesicular structures in the nanometer range and arise through invaginations of so-called multivesicular bodies and secretion by immune cells.

A “blood composition” means in the present case a composition of blood, in particular consisting of blood plasma, serum and blood cells, which comprises at least one component which is selected from proteins such as factors and cytokines. In the present case, a blood composition also means a blood serum composition. A blood serum composition differs from a blood composition in particular in that the blood serum composition does not (any longer) comprise cellular constituents. A conditioned blood serum composition is obtained from a conditioned blood composition obtainable according to the invention for example by removing the cellular constituents by centrifugation, filtration or other suitable measures from the blood composition, so that a cell-free solution of blood plasma and serum constituents which comprises at least the induced factors and cytokines is obtained.

In a preferred embodiment, accordingly, the cellular constituents are completely or substantially completely removed from the resulting conditioned blood composition in a further step, so that a conditioned blood serum composition is obtained. The conditioned blood serum can be employed like the blood composition obtainable according to the invention and usually confers the same technical advantages according to the invention. The skilled person will employ the conditioned blood serum composition or the conditioned whole blood composition according to the area of application and as expedient. He will preferably employ the conditioned blood serum composition.

The incubation of the blood in the at least one modified vessel is preferably continued until induction of the factors or cytokines has proceeded sufficiently far. The induced factors or cytokines are produced and secreted by the blood tissue substantially from the time when the incubation starts, so that an effective amount of the induced factors or cytokines accumulates in the conditioned blood composition.

In one embodiment of the invention, the appearance of IL-6 in the blood composition indicates successful and sufficiently further advanced induction. The proportion of IL-6 in this connection is in particular at least 30 pg/ml. Incubation is carried out in the modified vessel preferably until at least 30 pg/ml IL-6 are present in the blood composition. In further preferred variants, incubation is continued until at least 200 pg/ml, preferably 500 pg/ml, particularly preferably 1000 pg/ml, are present in the blood composition.

In a further embodiment, incubation is carried out for a period of 36 hours or less. In a further embodiment, incubation is carried out for a period of 9 hours or less. In a further variant, incubation is carried out for a period of 2 or more and up to 36 or less, preferably up to 9 or fewer hours.

In a further embodiment, the incubation of the blood takes place under a low oxygen partial pressure (pO₂). The oxygen partial pressure during the incubation is in particular less than 5 kPa, preferably less than 3 kPa. In a preferred variant, the incubation of the blood takes place in the modified vessel with exclusion of oxygen.

In a preferred embodiment, the modified vessel has in its interior particular structures with a large surface area, so that the internal surface area primarily resulting from the (external) geometry of the vessel is enlarged by the particular structures. The surface area enlargement by the particular structures is preferably from 10% to about 200%, in one variant from 10% to 100%. These preferably include structures with a large surface area/volume ratio such as spheres and fibers, but also other particles such as flour and granules, or combinations of such structures. The surface of these structures is preferably smooth. As an alternative it is possible in some cases to employ structures with a rough surface.

The skilled person will chose the number and shape of the internal structures according to the area of application and as expedient. It is self-evident that the shape and number of the internal structures to be added is chosen in this connection so that the total of the surface area of the added internal structures and of the internal surface of the vessel to be modified is matched in such a way that the surface area/volume ratio (surface index) intended according to the invention is obtained.

The modified vessel preferably has a non-pyrogenic internal surface. The modified vessel is preferably composed of pyrogen-free material.

If particulate internal structures such as spheres, fibers, flour, granules or mixtures thereof are employed, they comprise or consist preferably of materials selected from metals, metal oxides or plastics and mixtures thereof. Preferred examples thereof are glass, corundum, quartz, polystyrene, polyvinyl chloride, polyethylene and polypropylene, and mixtures thereof. Borosilicate glass is particularly preferred. These materials are preferably pyrogen-free.

The at least one modified vessel preferably comprises in its interior glass spheres, particularly preferably of pyrogen-free borosilicate glass, where the glass spheres have an (average) diameter of from 0.5 to 5 mm, preferably 1.5 mm, 2.5 mm or 3.5 mm. The glass spheres are particularly preferably added to the vessel to be modified, depending on the receiving capacity of the vessel, in a number of about 10 to 500. If a vessel is intended for example to receive about 50 ml of blood, then preferably about 30 to 300 spheres, particularly preferably about 50 to 250 spheres, which have a diameter of, preferably, 3.5 mm, are introduced.

In a particularly preferred embodiment, a vessel preferably known from transfusion medicine is used to take blood and to store blood, such as syringe, blood tube or blood bag, which is modified by adding a certain proportion of such internal structures so that a modified vessel with an enlarged internal surface area is obtained. The invention accordingly provides the use of at least one modified vessel with a large internal surface area with the surface index according to the invention, which comprises internal structures selected from spheres, fibers, flour, granules, particles or combinations thereof, for producing a conditioned blood composition.

The skilled person can of course also take other or additional measures in order to obtain a modified vessel with enlarged internal surface area which can be employed according to the invention. In a further preferred embodiment, a vessel whose inner vessel walls has protuberances, cavities and/or projections, so that the surface area/volume ratio (surface index) intended according to the invention is reached.

In a preferred embodiment, the modified vessel has elastic vessel walls which preferably make it possible to remove blood air-free from the animal or human body, when the modified vessel which is essentially still empty of air expands only when the blood flows in, so that no unwanted air space can form in the vessel. It is self-evident that the number and surface area of the internal structures provided in the modified vessel to enlarge its internal surface area is governed not by the maximum capacity of the elastic vessel but, on the contrary, by the volume of the blood composition to be incubated.

Such an elastic vessel is preferably selected from blood bags provided in transfusion medicine, which are preferably single, double, triple or multiple bag systems. Whereas a single bag system is distinguished by usually having at least one opening for filling and emptying, double, triple and multiple bags represent arrangements of a plurality of bags which communicate with one another and are preferably in contact with one another via a tubing connection. Such bags are preferably constructed in a simple manner from two elastic sheets welded together.

In a particularly preferred embodiment, the at least one modified vessel employed according to the invention is a blood bag or blood bag system which has been modified by introducing a number and type of particles chosen according to the invention, preferably glass spheres.

The vessel is preferably a bag system as is used as two-chamber blood bag system for centrifuges for separating blood constituents in freshly removed blood. If the vessel is modified according to the invention, preferably by introducing glass spheres, the method according to the invention for producing a conditioned blood composition can be carried out therein. Subsequently, the blood components from the conditioned blood are fractionated in the blood bag system from which a conditioned blood serum composition free of “solid” blood constituents is obtained.

A preferred two-chamber blood bag system includes at least one primary vessel and at least one secondary vessel which form a communicating vessel system. Primary vessel and secondary vessel are connected by at least one, in particular closable, transfer line. In connection with the present invention, a “primary vessel” preferably means a vessel, that is to say container, in which the blood composition which is to be conditioned and subsequently where appropriate fractionated into its individual components is introduced, incubated and where appropriate subjected to a first fractionation. It is particularly preferred for primary vessel, secondary vessel and transfer line expediently to be fixed on a support plate. The transfer line is particularly preferably closable by at least one interruption which can be designed as valve, cog and/or stopper. A “secondary vessel” means a preferably vessel, that is to say container, in which the liquid or suspension which has optionally been completely or partly fractionated into its individual components in the primary vessel is completely or partly introduced and subjected to a second fractionation. Each of these vessels is preferably provided with in each case at least one, in particular closable, outflow and/or inflow line, in particular for supplying, that is to say introducing or reapplying, blood components and/or discharging, that is to say removing, blood components. The additional internal structures according to the invention, such as glass spheres, are preferably provided in the primary vessel, or introduced therein.

In a preferred embodiment, the bag or the blood bag system for removing the solid blood constituents from the conditioned blood serum composition by centrifugation is inserted into a centrifuge cup. This preferably has a configuration such that the vessel which is preferably in the form of an elastic bag is stretched during the centrifugation so that the vessel walls make partial and/or complete contact with the inner wall of the centrifuge cup. The use of a sterile cup is preferred. The tensile stress on the vessel walls and the contained cells during the centrifugation is particularly advantageously reduced thereby. The preferred use of a centrifuge cup also allows the use of mechanically lighter, thinner and less stable wall material for the preferred elastic bag.

In a preferred embodiment of the method, the incubated blood composition is an allogeneic blood composition, preferably a blood composition which is removed in the form of whole blood from a human or animal donor and, after the method according to the invention has been carried out, can be administered as conditioned blood composition, preferably as conditioned blood serum composition, to a human donor. In a variant, the blood composition is autologous, that is to say donor organism and recipient organism are identical. In this particularly preferred variant, all the advantages of the autologous donation can apply. The skilled person will choose the nature and identity of the donor depending on the use and as expedient. It is possible in this connection in general to consider the known criteria and advantages relevant for the choice of an autologous donation.

In an alternative variant, the blood composition is xenogeneic. This means that it is taken from an organism of a different species. For this purpose, the unconditioned blood composition is taken from an animal donor organism, for example a pig, in the form of whole blood and, after the method according to the invention has been carried out, the conditioned blood composition is administered to the individual to be treated which belongs to a different species, for example horse, human or sportsperson.

A further aspect of the invention is the provision of a conditioned blood composition which can be produced, or preferably is produced, by the method according to the invention. This composition can be employed according to the invention for the treatment, alleviation, cure or prevention of a disorder of the human or animal body. This blood composition comprises according to the invention the factors induced on carrying out the method of the invention, at least 30, preferably more than 200, 1000, 5000, 10 000 pg/ml, preferably from 30 to 20 000 pg/ml, interleukin-6. It is self-evident that the conditioned blood composition which can be produced by the method of the invention includes further induced factors besides interleukin-6 as one of the induced factors. It has surprisingly been possible to show that the composition of induced factors which is obtainable according to the invention in a cocktail precisely exhibits the advantages and effects according to the invention.

A preferred conditioned blood composition includes besides interleukin-6 (IL-6) at least one further component which is selected from: interleukin-1 receptor antagonist (IL-1Ra), interleukin-4 (IL-4), interleukin-13 (IL-13), interleukin-1 (IL-1), interleukin-10 (IL-1), tumor necrosis factor (TNF), insulin-like growth factor (IGF), transforming growth factor (TGF), platelet-derived growth factor (PDGF), fibroblast growth factor (FGF) and hepatocyte growth factor (HGF).

In one variant, the conditioned blood composition comprises interleukin-1 receptor antagonist (IL-1Ra) in a proportion of from 30 to 50 000 pg/ml. In a further variant, the conditioned blood composition comprises interleukin-4 (IL-4) in a proportion of from 2 to 100 pg/ml. In a further variant, the conditioned blood composition comprises interleukin-13 (IL-13) in a proportion of from 2 to 100 pg/ml. In a further variant, the conditioned blood composition comprises interleukin-1 (IL-1) in a proportion of from 5 to 1000 pg/l. In a further variant, the conditioned blood composition comprises interleukin-10 (IL-10) in a proportion of from 5 to 1000 pg/l. In a further variant, the conditioned blood composition comprises tumor necrosis factor (TNF) in a proportion of from 5 to 1000 pg/l. In a further variant, the conditioned blood composition comprises insulin-like growth factor (IGF) in a proportion of from 100 to 15 000 pg/ml. In a further variant, the conditioned blood composition comprises transforming growth factor (TGF) in a proportion of from 10 to 20 000 pg/ml. In a further variant, the conditioned blood composition comprises platelet-derived growth factor (PDGF) in a proportion of from 100 to 10 000 pg/ml. In a further variant, the conditioned blood composition comprises fibroblast growth factor (FGF) in a proportion of from 50 to 10 000 pg/ml. In a further variant, the conditioned blood composition comprises hepatocyte growth factor (HGF) in a proportion of from 50 to 10 000 pg/ml.

Surprisingly, the cocktail of induced factors and cytokines present in the conditioned blood composition obtainable according to the invention has particularly efficient prophylactic and therapeutic effects. The conditioned blood composition or the conditioned blood serum composition obtainable therefrom is employed according to the invention particularly effectively for a number of diseases or disorders of the human or animal body, which are treated, cured or alleviated therewith, or with which these diseases and disorders is prevented.

The conditioned blood composition or blood serum composition obtainable according to the invention is employed according to the invention for muscle disorders, for disorders of the musculoskeletal system as well as inflammations and irritations of the nervous system, especially disorders of the tendon system such as tendon injuries, tenosynovitis, ligament injuries, tendon degeneration and ligament degeneration, and for rapid cure, alleviation or prevention of allergies, food or drug intolerances, disorders involving the immune system, especially autoimmune diseases, especially rheumatoid diseases, and disorders caused by neurodermatitis, and for the treatment and healing of chronic wounds, especially diabetic ulcers, the treatment of endometriosis, and the treatment of chronic eye inflammation and regeneration or improvement of pain from irritation of the tendons in horses. The muscle disorders include muscle disorders arising through muscle injuries associated with muscle operations, in connection with muscle fiber tears associated with muscle degeneration, with muscle defects, with muscle atrophy, with myocele, with muscular dystrophy, or are attributable to muscle fatigue or muscular soreness.

The present invention therefore relates to the use of the blood composition according to the invention of the conditioned blood serum composition obtainable therefrom for the treatment or prevention of a disorder of the human or animal body. The disorder of the human or animal body which is preferably treated, alleviated or cured or which can be prevented is selected from rheumatoid diseases, diseases of the musculoskeletal system, and diseases associated with the immune system, and diseases which cause acute or chronic pain.

It is self-evident that the skilled person will choose the mode of administration which is expedient in each case for administering the conditioned blood composition according to the invention for appropriate treatment of the respective disorder. The conditioned blood composition or blood serum composition is preferably injected or infused into the body and/or the affected organ such as joint, muscle, tendon, skin or nerve, preferably intravenously, intraarterially, subcutaneously, intradermally, subconjunctivally, topically, intrathecally, perispinally, into and/or onto central nerves, into and/or onto peripheral nerves, intraarticularly and/or intramuscularly.

In a further aspect of the invention, the conditioned blood composition according to the invention is therefore used to produce a medicament for the treatment or prevention of a disorder of the human or animal body. These disorders are characterized above.

Besides this, one use of the blood composition according to the invention is also provided as non-therapeutic cosmetic, as so-called anti-aging agent. It has emerged that physical manifestations associated with age, especially the aforementioned symptoms, can be alleviated or cured or else the external appearance of skin, hair, nails can be improved by systematic and/or topical administration. In a further aspect of the invention, the blood composition according to the invention is used to produce cosmetics.

Finally, the present invention also relates to a method for the treatment or prevention of a disorder, characterized above, of the human or animal body, which comprises at least the following step: administration of the blood composition conditioned according to the invention to the human or animal body in a therapeutically or prophylactically effective dose. The dose and mode of administration will be chosen by the skilled person according to the area of application and as expedient.

### EXEMPLARY EMBODIMENTS

The invention is explained in more detail by the following examples and the FIGURE, but the examples are not to be understood as restrictive. The skilled person will realize the basic principle of the invention and the technical advantages connected therewith from the examples. He will be able to apply the basic principles and technical advantages to other sectors not expressly mentioned here.

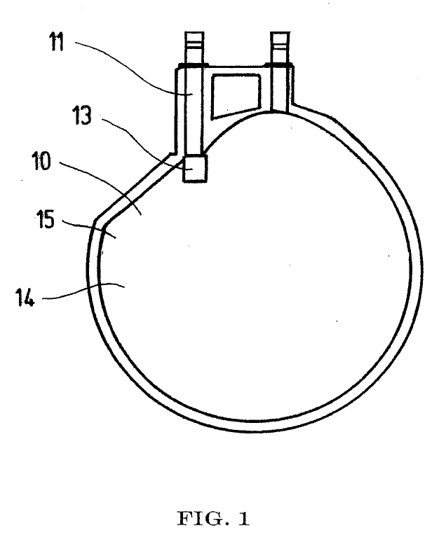

FIG. 1 shows a diagrammatic representation of a preferred embodiment of the apparatus of the invention, consisting of a vessel (10) configured as elastic bag and having a preferably semicircular lower section (14) and a preferably tapering upper section (15) with at least one inflow and/or outflow line (11) which opens into the funnel-shaped upper section (15) of the vessel. At its lower end, which opens into the lumen of the vessel 10, a spear valve (13), that is to say shutter valve, is preferably provided.

### EXAMPLE 1

### Kit for Obtaining Conditioned Blood Composition from Whole Blood

A sterilizable single-use kit is assembled and comprises the following:
a bag system for incubating the blood and for removing solid blood constituents (FIGS. 1 and 2, table 1), equipped with borosilicate glass spheres (about 200) with a diameter of 3.5 mm,
20-gauge needle for drawing anticoagulant into the blood-collecting syringe,
60 ml syringe for collecting blood,
butterfly needle for collecting blood,
60 ml syringe to receive the conditioned blood serum composition

All the components are single-use articles, packaged and gamma-sterilized and provided as a whole with sterile outer pack.

Tables 1 and 2 list the materials of the components used.

TABLE 1

| Component | Material, supplier |
|---|---|
| Bag (10) | Bag film: PVC compound 3222 (Solvay Draka) |
| Internal | 200 borosilicate glass spheres, 3.5 mm |
| structures | diameter (Duran ®) |

TABLE 2
Kit for producing a conditioned blood composition

| Component | Material, supplier | |
|---|---|---|
| apparatus of the | (see Table 1) | |
| invention | | |
| Butterfly needle 1.1 × | closure cap: | PE |
| 19 m | LL adapter: | ABS transparent |
| | winged connecting head: | PVC |
| | tubing: | PVC 60 Sh A |
| | needle: | ISO 638/13 |
| | protective tubing: | PE |
| needle 1.1 × 40 mm | connecting head: | PP |
| | protective cap: | PP |
| | needle: | stainless steel |
| | | complying with |
| | | DIN EN ISO 9626 |
| 60 ml syringe | barrel: | PP |
| | plunger shank: | PP |
| | plunger head: | natural rubber |
| 10 ml syringe (12 cc) | barrel: | PP |
| | plunger shank: | PP |
| | plunger head: | PP |
| Perfusor line 1.5 m, | LL male: | ABS KR 2802 |
| 1.0 × 2.7 mm | cap: | PE, opaque |
| | LL female: | PVC |
| | cap: | ABS, red |
| | tubing: | |
| | inner layer: | ND, PE |
| | middle layer: | EVA |
| | outer layer: | PVC |
| Blister pack | PET-GAG | |
| 0.9 × 206 × 500 mm | | |
| Tyvek sealing paper | Tyvek 10MP/1073B | |

### EXAMPLE 2

### Obtaining a Conditioned Blood Serum Composition from Whole Blood

### a) Blood Collection

The blood is collected with a 60 ml Luer lock syringe. The syringe is slowly filled to the 60 ml mark with whole blood. Care is taken that filling is bubble-free so exactly 60 ml are in fact present in the syringe.

### b) Charging the Bag System and Incubation

The contents of the syringe are slowly and completely introduced via the inflow/outflow line (11) into the vessel (10) which is configured as elastic bag. The vessel already contains about 200 spheres of borosilicate glass (Duran®) diameter 3.5 mm.

After the charging, the syringe is unscrewed and the connector (12) of the bag is reclosed with a new closure cap.

The vessel (10) is stored, preferably suspended, at about 37° C. for 9 to 36 hours. During this, the removed blood is incubated in the vessel with the spheres which enlarge the internal surface area. The enlarged internal surface area is about 350 mm² per 1 ml of incubated blood.

### c) Removal of the Solid Constituents

The vessel (10) is inserted into a centrifuge cup in a sterile centrifuge suspension gear. After a check of the correct weight distribution, the centrifugation is carried out at about 2500 rpm for about 3 min. After completion of the centrifugation, in which separation of the cellular from the liquid blood constituents takes place, the centrifuge cup is carefully removed together with the vessel (10).

Blood cells, mainly erythrocytes (EC) have collected in the lower section of the vessel (10) owing to the centrifugation. The centrifugation separates serum from blood clot. The serum is transferred into the second bag and then centrifuged a second time where appropriate. The conditioned serum composition is removed through the removal connector of the inflow/outflow line (11). The filled syringe is then unscrewed.

### EXAMPLE 3

### Analysis of the Conditioned Blood Serum Composition

Four test batches A, B, C, D and E were produced and were used in the same way for incubating whole blood.

In a batch A, a commercially available blood bag (OSTEOKIN, Orthogen, Düsseldorf) which is essentially described in Examples 1 and 2 was charged with 210 spheres of borosilicate glass (Duran®) with a diameter of 3.5 mm. Owing to the addition of the internal structures, the internal surface area of the modified vessels totals about 18 125 mm². On incubation of 50 ml of blood, the surface area/volume ratio (surface index) of the modified vessel is about 360 mm²/ml.

In a further batch B, the same blood bank system as in batch A was employed, but no additional internal structures were introduced. The uncharged blood bag system has an internal surface area of about 10 000 mm². With 50 ml of blood, this corresponds to a surface index of about 200 mm²/ml.

In a further batch C, the same blood bag system as in batch A was employed and was charged with 780 glass spheres with a diameter of 3.5 mm. The internal surface area then totals about 40 000 mm². The surface index when charged with 50 ml of blood is about 800 mm²/ml.

In a further batch D, a different blood removal system which has an essentially cylindrical shape was charged with 36 glass spheres with a diameter of 1.5 mm. The internal surface area then totals about 4050 mm². The surface index when charged with 10 ml of blood is about 405 mm²/ml.

In a further batch E, a blood removal system which has an essentially cylindrical shape was charged with 62 glass spheres with a diameter of 3.5 mm. The internal surface area then totals about 6200 mm². The surface index when charged with 10 ml of blood is about 620 mm²/ml.

In all the test batches, venous whole blood was in each case freshly removed and in each case introduced into the vessels of batches A, B and C. The vessels were incubated at about 37° C. for 24 hours (t=24 h). In addition, as control, in each case about 10 ml of fresh whole blood from the same donors was worked up directly after the blood was taken (t=0 h).

After the incubation time had elapsed, the blood components IL-1Ra, IL-6, TNFa and IL-1β in the blood compositions were quantified.

Results: Table 3 shows the results.

TABLE 3

| Factor/ | | t = 24 h | | | | |
|---|---|---|---|---|---|---|
| cytokine | | A | B | C | D | E |
| [pg/ml] | t = 0 h | 360 mm²/ml | 200 mm²/ml | 800 mm²/ml | 405 mm²/ml | 620 mm²/ml |
| IL-1Ra | 323.7 | 8592 | 6626 | —* | 2663 | 7836 |
| IL-6 | 3.7 | 2830 | 1571 | —* | 847.5 | 2933 |
| TNFa | 19.9 | 718.3 | 204.5 | —* | 31.54 | 569.8 |
| IL-1β | 1.00 | 396.6 | 92.69 | —* | 16.81 | 154.8 |

*cytolysis, no measurement

Whereas batches A and B showed a marked induction of the analyzed factors in the blood composition, hemolysis occurred during incubation of batch C. It emerges that the strength of induction depends on the surface index: with a larger surface index (larger internal surface area) a larger proportion of induced cytokines is obtained. At the same time there is an upper limit of the surface index; if a critical value is exceeded, hemolysis occurs. A hemolyzed blood composition cannot be used further. With large surface indices near the critical value, the hemolysis can be suppressed within certain limits by shortening the incubation time from 24 hours to 6 to 9 hours (data not shown).

### EXAMPLE 4

### Cytokine Profile of the Conditioned Blood Composition

In a further batch, 36 glass spheres of borosilicate glass (Duran®) with a diameter of 1.5 mm were introduced into a cylindrical blood removal vessel to enlarge the internal surface area. 50 ml of freshly removed whole blood were incubated. The surface index was about 405 mm²/ml.

Blood was incubated in the blood removal vessel at about 37° C. for three hours, nine hours and 24 hours. The content of the cytokines FGF, IL-4, IL-10, IL-1β, TNF, IL-6, IL-1Ra and TGFβ was then determined.

### Results:

There was a marked rise in the cytokine content in the conditioned blood composition after incubation for only three hours. Table 4 compares the values measured after hours (t=24 h) with the values measured directly after removal of the blood (t=0 h).

TABLE 4

| Factor/cytokine | | |
|---|---|---|
| [pg/ml] | t = 0 h | t = 24 h |
| FGF | 0.1 | 2.0 |
| IL-4 | 5.4 | 7.9 |
| IL-10 | 7.9 | 55.4 |
| IL-1β | 3.9 | 409 |
| TNF | 6.0 | 536 |
| IL-6 | n.a. | 3444 |
| IL-1Ra | 241.9 | 9975 |
| TGFβ | 18313 | 36696 |

### EXAMPLE 5

### Treatment of Neurodermatitis

Neurodermatitis was treated by administering the conditioned blood composition produced according to the invention to patients in the form of injections, also as intraarticular injections. This entailed 2 ml of the conditioned blood composition being injected at an interval of 2 to 3 days in each case over a period of 3 weeks. It was possible to find an improvement in the symptoms of neurodermatitis within 3 days. A renewed flair up of the disorder after about 2.5 months was likewise successfully treated with 3 injections.

In other patients for whom intraarticular injections were employed primarily for the treatment of their knee pain (caused by arthrosis and discomfort in the meniscus), the neurodermatitis symptoms also improved over the course of 6 injections. Since then, no flair up of the neurodermatitis has occurred.

### EXAMPLE 6

### Treatment of Inflammation or Irritations of the Nervous System

In this application of the conditioned blood composition produced according to the invention, patients with backache (n=30) who had suffered chronically for at least 6 months from radicular-related backache were treated by local injections at the nerve root (epidural-peridural injection according to Kramer et al.). The pain improved within a few weeks, and the effect was on average still manifest after 6 months. The result in this case was at least equivalent or slightly improved by comparison with patients treated with the same injection technique with either 5 mg or 10 mg of glucocorticoid (triamcinolone) as comparative substance.

### EXAMPLE 7

### Treatment of Endometriosis

Patients (n=4) suffering from painful endometriosis were treated by one intraperitoneal injection of 4 ml of the conditioned blood serum produced according to the invention directly into the neoplastic tissue caused by the endometriosis and/or into the abdominal cavity. These administrations were initially accompanied by severe pain but were followed within a few hours by marked reduction in the pain. This effect persisted and led to almost complete freedom from pain on the following day. The therapy was continued by further treatment at weekly intervals with subcutaneous injections of in each case 2 ml of the conditioned blood serum. No relapse or recurrence of pain has been observable to date. The pain-relieving effect of the blood composition conditioned according to the invention surprisingly goes far beyond the effect of normal analgesics.

### EXAMPLE 8

### Chronic Eye Inflammation in Horses

The conditioned blood composition produced according to the invention was used to treat chronic eye inflammation in horses (equine recurrent uveitis, ERU) by (subconjunctival) injection into the eye, or drops (topical) in the eye, of 6 horses, of which 3 horses in each case were treated in two different veterinary practices. No relapse was found in any of the treated cases within the follow-up period of up to 10 months.

### EXAMPLE 9

### Regeneration and Improvement in Pain from Tendon Irritations in Horses

In a further application of the conditioned blood composition, horses with lameness caused by extensive inflammation or irritation of the tendon sheath associated with effusion into the tendon sheath were treated with injections of 3 ml in each case of the conditioned blood serum according to the invention into the tendon sheath. For this purpose, initially, in a first step the effusion was tapped in order to reduce the pressure on the tissue and to remove proinflammatory substances. After the first injection there were marked reductions both in the lameness within one week and in the amount of effusion detectable in the second week. After 4 weeks, that is to say one week after injection of the third and last dose into the tendon sheath, there was found to be almost complete remission both of the lameness and of the effusion.

Similar injections into so-called core lesions and/or superficial lesions, that is to say degenerative changes within the tendon sheath, likewise led to a marked remission of these clinical symptoms. In some cases, the defect was observed to be refilled with collagen fibers.

### EXAMPLE 10

### Treatment of Wounds in Horses

A 14-year old gelding with lameness in several joints had suffered for many weeks from a persistent wound above the left forehoof of the hoof. The conditioned blood composition according to the invention was applied as drops to this wound drops on an area of about 1×3 cm). The wound was then dressed. After the concluding inspection (after three treatments at weekly intervals) after a period of 4 weeks it was found that the open wound area had reduced by about one-third.

1. A method for producing a conditioned blood composition from blood, which method comprises the following steps:
(a) removing blood from a human or animal body,
(b) incubating the removed blood in a modified vessel with an internal surface area at a temperature of from 10 to 40° C. to condition the blood, with induction of factors, and where the modified vessel has an internal surface area of from 200 mm² to 750 mm² per 1 ml of incubated blood; and
(c) obtaining a conditioned blood composition with induced factors in the modified vessel.

2. The method as claimed in claim 1, where the occurrence of interleukin-6 (IL-6) in the blood composition in a proportion of at least 30 pg per 1 ml indicates successful induction.

3. The method as claimed in claim 1, wherein incubation occurs for a period of from 2 to 36 hours.

4. The method as claimed in claim 1, where oxygen partial pressure (pO₂) during the incubation is less than 5 kPa.

5. The method as claimed in claim 1, wherein in a further step cellular constituents are removed from the conditioned blood composition, and a conditioned blood serum composition is obtained thereby.

6. The method as claimed in claim 1, where the modified vessel has internal structures with large surface areas and wherein the internal structures are selected from spheres, fibers, flour, granules, particles and combinations thereof.

7. The method as claimed in claim 6, where the internal structures are comprised of at least one material selected from metal, metal oxide, plastics, and combinations thereof.

8. The method as claimed in claim 1, where the modified vessel contains in its interior glass spheres which have a diameter of from 0.5 to 5 mm.

9. The method as claimed in claim 1, where the modified vessel has elastic vessel walls for removing to permit removal of blood air-free from the animal or human body.

10. The method as claimed in claim 9, where the vessel is selected from blood bags for transfusion medicine.

11. The method as claimed in claim 10, where the vessel is selected from the group consisting of single, double, triple and multiple bag systems.

12. The method as claimed in claim 9, where the elastic vessel walls have a low oxygen permeability.

13. The method as claimed in claim 1, where the blood composition is allogeneic.

14. The method as claimed in claim 1, where the blood composition is autologous.

15. The method as claimed in claim 1, where the blood composition is xerogenic.

16. A blood composition produced by the method as claimed in claim 1, adapted for the treatment or prevention of a disorder of the human or animal body, comprising 30 to 20,000 pg/ml interleukin-6 (IL-6).

17. The blood composition as claimed in claim 16, comprising at least one further component selected from:
interleukin-1 receptor antagonist (LI-1Ra),
interleukin-4 (IL-4),
interleukin-13 (IL-13),
interleukin1 (IL-1),
interleukin 10 (IL-10),
tumor necrosis factor (TNF),
insulin-like growth factor (IGF),
transforming growth factor (TGF),
platelet-derived growth factor (PDGF),
fibroblast growth factor (FGF), and
hepatocyte growth factor (HGF).

18. The blood composition as claimed in claim 16, further comprising at least one component selected from vesicles, microvesicles, exosomes, iRNA and mixtures thereof.

19. The blood composition as claimed in claim 16, where interleukin-1 receptor antagonist (IL-1Ra) is present in an amount of 30-50,000 pg/ml.

20. The blood composition as claimed in claim 16, where interleukin-4 (IL-4) is present in an amount of 2-100 pg/ml.

21. The blood composition as claimed in claim 16, where interleukin-13 (IL-13) is present in an amount of 2-100 pg/ml.

22. The blood composition as claimed in claim 16, where interleukin-1 (IL-1) is present in an amount of 5-1000 pg/ml.

23. The blood composition as claimed in claim 16, where interleukin-10 (IL-10) is present in an amount of 5-1000 pg/ml.

24. The blood composition as claimed in claim 16, where tumor necrosis factor (TNF) is present in an amount of 5-1000 pg/ml.

25. The blood composition as claimed in claim 16, where insulin-like growth factor (IGF) is present in an amount of 100-15,000 pg/ml.

26. The blood composition as claimed in claim 16, where transforming growth factor (TGF) is present in an amount of 100-20,000 pg/ml.

27. The blood composition as claimed in claim 16, where platelet-derived growth factor (PDGF) is present in an amount of 100-10,000 pg/ml.

28. The blood composition as claimed in claim 16, where fibroblast growth factor (FGF) is present in an amount of 50-10,000 pg/ml.

29. The blood composition as claimed in claim 16, where hepatocyte growth factor (HGF) is present in an amount of 10-10,000 pg/ml.

30. A method for the treatment or prevention of a disorder of the human or animal body, said disorder selected from the group consisting of:
muscle disorders,
disorders of the tendon system,
allergies,
food intolerances,
disorders involving the immune system,
psoriasis and
chronic wounds such as diabetic ulcers, wherein the method compromises administering to one afflicted with or who may be afflicted with at least one said disorder a sufficient amount of the blood composition as claimed in claim 16 to respectively treat or prevent said disorder.

31. The method as claimed in claim 30, where the muscle disorder is a muscle injury, a muscle operation, a muscle fiber tear, a muscle degeneration, a muscle defect, a muscle atrophy, a myocele, a muscular dystrophy, a muscle fatigue or muscle soreness.

32. The method as claimed in claim 30, where the treatment of a muscle disorder includes regeneration of muscle tissue.

33. A method for the treatment or prevention of a disorder of the human or animal body, said disorder selected from the group consisting of:
neurodermatitis,
inflammations and irritations of the nervous system,
endometriosis, and
chronic eye inflammation in horses, wherein the method compromises administering to a subject afflicted with or who may be afflicted with said disorder a sufficient amount of the blood composition as claimed in claim 16 to respectively treat or prevent said disorder.

34. The method as claimed in claim 30, where the blood composition is injected where appropriate together with pharmaceutical excipients into the body or affected organ.

35. A method for the production of a medicament for the treatment or prevention of a disorder of the human or animal body according to claim 30, wherein the method comprises including in said medicament a therapeutic or preventative amount of the blood composition as claimed in claim 16.

36. A method of forming a cosmetic, wherein the method comprises including in said cosmetic the blood composition as claimed in claim 16.

37. The method as claimed in claim 7, wherein the internal structures are comprised of a metal oxide material selected from the group consisting of glass, corundum and quartz.

38. The method as claimed in claim 7, wherein the internal structures are comprised of a plastic material selected from the group consisting of polystyrene, polyvinyl chloride, polyethylene and polypropylene.
